(19) United States
(12) Patent Application Publication
(10) Pub. No.: US 2022/0218276 A1
(43) Pub. Date: Jul. 14, 2022

(54) ANESTHESIA MONITORING DEVICE AND SYSTEM

(71) Applicants: Paul Barton, Boerne, TX (US); Inform Wayland d.o.o., 1000 Skopje (MM)

(72) Inventors: Paul BARTON, Boerne, TX (US); James F. Anderson, Skopje (MK)

(21) Appl. No.: 17/571,367

(22) Filed: Jan. 7, 2022

Related U.S. Application Data
(60) Provisional application No. 63/135,358, filed on Jan. 8, 2021.

(51) Int. Cl. A61B 5/00; G16H 40/63

(52) U.S. Cl. CPC A61B 5/4821; A61B 5/7203; A61B 2560/0214; G16H 40/63; A61B 5/0022

(57) ABSTRACT

A data collecting device including a microcontroller communicatively coupled to at least one of a plurality of sensors, the sensors being configured to detect vital signs of a patient including at least one of temperature, blood oxygen saturation level, blood pressure, and a level of carbon dioxide exhaled by the patient, the microcontroller being configured to, in response to receiving data from at least one of the sensors, perform noise filtering and aggregate the received sensor data into a plurality of data packets, and transmit the filtered and aggregated data to portable mobile computing device communicatively coupled thereto.

### REFERENCE TO RELATED APPLICATION

This application claims benefit of priority from U.S. Provisional Application 63/135,358, filed Jan. 8, 2021, the disclosure of which is hereby incorporated in its entirety by reference herein.

### TECHNICAL FIELD

This invention relates to systems and methods for monitoring data related to one or more physiological parameters of a patient under surgical anesthesia and/or during general patient monitoring in clinical settings which lack reliable infrastructure.

### BACKGROUND

Surgical procedures often require general anesthesia. To minimize patient risk during anesthesia, certain patient vital signs may be monitored to ensure that the vital signs stay within acceptable ranges and to quickly respond to any deviation from acceptable ranges.

Existing vital signs monitoring devices used by anesthesiologists generally have integral displays and are powered from the power mains, making them expensive, less portable, less durable, and particularly unsuitable for use in harsh conditions or remote areas lacking reliable infrastructure. Consequently, otherwise necessary surgical procedures may not be attempted, because administering general anesthesia without the ability to adequately monitor patient vital signs may pose inappropriate risk to the patient. Given the high cost of medical equipment, it is highly desirable for equipment to be easily transportable from location to location, so doctors can carry their own monitoring equipment with them.

### SUMMARY

Accordingly, an example embodiment of a screenless anesthesia monitoring system may be constructed for use in monitoring a selected plurality of physiological conditions of a patient in a medical environment. In some instances, the screenless anesthesia monitoring system includes one or more sensors, such as, but not limited to, sensors for detecting exhaled carbon dioxide amounts, a non-invasive blood pressure sensor, a sensor for measuring or determining specific blood oxygenation, body temperature, and cardiac rhythm (i.e. electrocardiogram (ECG or EKG)). Moreover, the screenless anesthesia monitoring system may include more or fewer sensors and/or different sensors.

As will be described in more detail in the following description, the screenless anesthesia monitoring system resolves a number of deficiencies found in prior art systems. The screenless anesthesia monitoring system is battery-powered and has no integrated user interface, instead utilizing, for example, one or more components of a mobile computing device to perform digital signal processing, display and store data, and communicates with one or more additional components, devices, or systems.

Being battery-powered, the screenless anesthesia monitor is not reliant on infrastructure and may be used in remote locations and under harsh conditions. Furthermore, being battery-powered enables the screenless anesthesia monitoring system to operate without electrical isolation that may be necessary for patient safety when a mains-powered equipment is used. Still further, the battery-powered screenless anesthesia monitoring system may be subject to smaller amounts of conducted electric and electromagnetic interference than equipment powered by mains power, resulting in a smaller, lighter, and less expensive device.

A mobile computing device of the screenless anesthesia monitoring system may be configured to communicate with one or more components or devices using one or more wired or wireless communication protocols, such as, but not limited to, short-range or ultra-short-range communication protocols, e.g., Bluetooth™ and near field communication (NFC).

Furthermore, not having a screen may make the screenless anesthesia monitoring device more resistant to moisture, dust, and impact than existing designs and makes it possible for standard user-replaceable AA alkaline batteries to provide long battery life.

The resulting system described in this application is low cost and very portable, enabling doctors in low income areas to more readily afford the devices and to be able to easily move those devices from location to location, where they can be used even in unfavorable conditions.

The advantages of the invention described herein will be evident from the detailed description of preferred embodiments of the invention that follows and in the accompanying drawings.

A data collecting device includes a microcontroller communicatively coupled to at least one of a plurality of sensors. The sensors are configured to detect vital signs of a patient including at least one of: cardiac rhythm, temperature, blood oxygen saturation level, blood pressure, and a level of carbon dioxide exhaled by the patient. The microcontroller is configured to, in response to receiving data from at least one of the sensors, perform noise filtering and aggregate the received sensor data into a plurality of data packets, and transmit the filtered and aggregated data to portable mobile computing device communicatively coupled thereto.

In some examples, the device includes a power source comprising one or more alkaline cells. In some other examples, the microcontroller is configured to cause the aggregated data to be cached in the mobile computing device, and to transfer the received sensor data to at least one of a computer cloud and a network drive.

A data collecting device includes a plurality of sensors configured to detect vital signs of a patient including at least one of: cardiac rhythm, temperature, blood oxygen saturation level, blood pressure, and a level of carbon dioxide exhaled by the patient. The data collecting device includes a microcontroller communicatively coupled to at least one of the sensors and being configured to, in response to receiving data from the at least one of the sensors, transmit the received sensor data to a mobile computing device communicatively coupled thereto.

In some examples, the data collecting device is configured such that, prior to transmitting the received sensor data, the microcontroller filters the received sensor data. In some other examples, the data collecting device is configured such that the microcontroller filters the received sensor data to remove 50 Hz and 60 Hz mains noise. In some other examples, the data collecting device is configured such that the microcontroller causes the aggregated data to be cached in the mobile computing device. In still other examples, the data collecting device is configured such that the microcontroller transfers the received sensor data to at least one of a computer cloud and a network drive. In yet other examples, the data collecting device includes a power source configured to power the microcontroller and the power source is a battery.

A method includes detecting, by a microcontroller of a screenless anesthesia monitoring device, a value of at least one vital parameter of a patient including at least one of: cardiac rhythm, temperature, blood oxygen saturation level, blood pressure, and a level of carbon dioxide exhaled by the patient, the microcontroller being communicatively coupled to at least one of a plurality of sensors, and the detecting the vital parameters including receiving signals from the at least one of the plurality of sensors including data indicating the value of the detected vital parameter. The method includes performing noise filtering and aggregate the received sensor data into a plurality of data packets. The method includes transmitting the filtered and aggregated data to a portable mobile computing device communicatively coupled to the screenless anesthesia monitoring device, wherein a processor of the screenless anesthesia monitoring device is configured to cause the value of the detected vital parameter to be displayed on a website page.

In one example, the method is such that filtering the received sensor data includes removing 50 Hz and 60 Hz mains noise. In another example, the method is such that the processor of the mobile communication device is configured to cache the aggregated data in a storage device of the mobile communication device. In still another example, the method includes transferring the received sensor data to at least one of a computer cloud and a network drive communicatively coupled to at least one of the screenless anesthesia monitoring device and the mobile communication device. In yet another example, the method is such that the microcontroller of the screenless anesthesia monitoring device is powered using a power source, and wherein the power source is a battery. In still another example, the method is such that the sensors include a corresponding one of a temperature measurement sensor, an electrocardiogram device, an oxygen saturation level measurement device, a non-invasive blood pressure measurement device, and a carbon dioxide measurement device.

### BRIEF DESCRIPTION OF THE DRAWINGS

The following figures illustrate the presented invention and are part of the specification:

FIG. 1 is a block diagram illustrating an example implementation of a screenless anesthesia monitoring device including sensing extensions and a mobile device in accordance with the present disclosure;

FIG. 2 is a block diagram illustrating an example implementation of a communication network including the screenless anesthesia monitoring device and the mobile device of FIG. 1; and

FIG. 3 is a block diagram illustrating an example implementation of the mobile communication device of FIG. 1.

Since is the figures are for illustrative purposes only, they represent implementations of the system of the present disclosure in a functional, rather than physical, sense. As such, the figures do not exclude different configurations or representations of the illustrated functional elements.

### DETAILED DESCRIPTION

Medical monitoring equipment that relies heavily on mains or electrical grid power may not be useful in many remote or other areas that have low quality or intermittent power. Loss of power during a surgical procedure may disable such medical monitoring equipment preventing collection of future data and causing a loss of historical data collected during the procedure. In hospitals that rely on AC generators or in areas with unstable electrical infrastructure, voltage fluctuations from rapidly changing electrical loads may damage or destroy expensive equipment. Patient-connected devices which are mains-powered require the use of fully isolated power supplies to ensure patent safety. Thus, AC power supplies contribute significantly to the size and cost of the medical monitoring equipment and may also contribute 50- or 60-Hz electrical noise, thereby complicating vital signs analysis.

The integrated nature of existing anesthesia monitoring equipment increases manufacturing costs, decreases portability, and increases susceptibility to physical damage Resulting equipment is often too expensive to allow the purchase of equipment for every location, and at the same time too large and fragile for doctors to easily transport a single device from one location to another.

Highly integrated medical equipment is typically costly since the equipment and design must undergo certification on all internal components, even those not directly contributing to patient safety must be of hospital grade and from a specific origin and of certified quality. Furthermore, obsolescence is a problem because components in a previously certified design may not continue to be available, forcing manufacturers to carry excessive component inventory, further increasing unit costs.

Integrated display screens increase the size of diagnostic and monitoring equipment making the equipment significantly less transportable. Further, because the display is often exposed and is one of the most fragile parts of the equipment, integrated displays increase possibility that the equipment may become inoperable or unusable.

For surgical and clinical use in remote locations or locations with inadequate infrastructure, it is desirable to have a highly portable device which can be purchased at low cost, can be used without constant mains power, can be easily upgraded to take advantage of new technology capabilities, and can be easily transported from location to location with increased protection from physical damage or harsh operating conditions.

Accordingly, there exists a need for a portable, modular system for monitoring vital signs of patients undergoing anesthesia, that for the purposes of this application is defined as a monitoring system which offers hospital-grade performance, is battery powered, and which utilizes an external mobile device for user interface and control and for the display, storage, processing, and communications of acquired vital signs data.

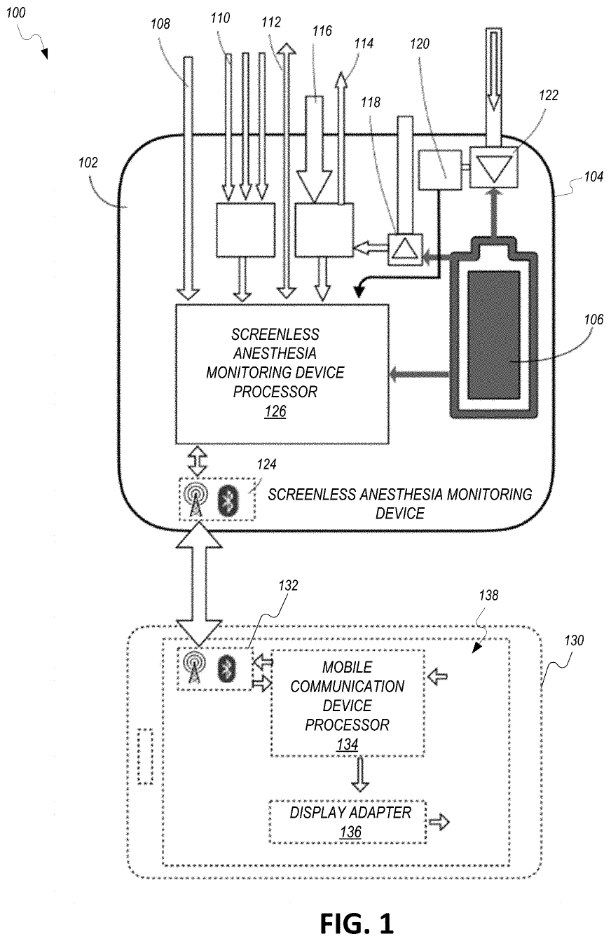

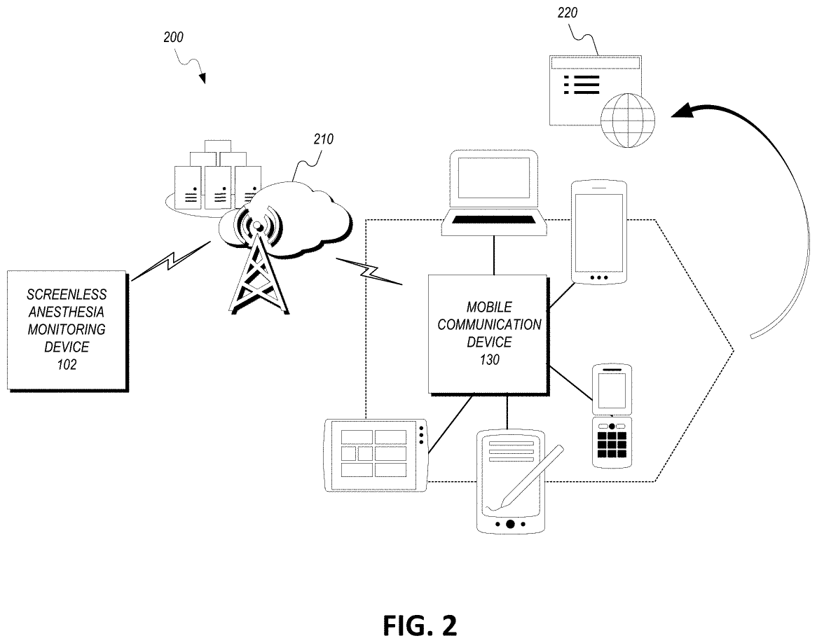

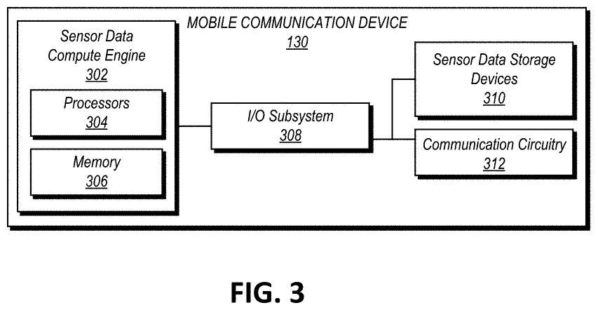

FIG. 1 illustrates an example monitoring system 100 including a screenless anesthesia monitoring device 102 in accordance with the present disclosure. The screenless anesthesia monitoring device 102 includes a plurality of sensors or devices, such as, but not limited to, electrodes, sensing pads, probes, leads, and wires, to detect, monitor, capture, transmit, and process patient physiological parameters during routine monitoring or when the patient is under a general anesthesia. As described in further detail in reference to FIGS. 2 and 3, the screenless anesthesia monitoring device 102 may be configured to be communicatively coupled to a mobile communication device 130 to transmit data or issue control commands to the mobile communication device 130 and to receive data or control commands from the mobile communication device 130. The arrows in the diagram of FIG. 1 represent one or more signal flow, control command flow, and/or data flow to, from, and between various components of the example monitoring system 100. The screenless anesthesia monitoring system, or SAM, 102 may be configured to monitor one or more patient vital signs, physiological parameters, or other values, levels, amplitudes, ranges, or thresholds useful or necessary during routine monitoring or when the patient is under a general anesthesia.

The screenless anesthesia monitoring device 102 includes a housing (also referred to as, electronic chassis) 104. The housing 104 of the screenless anesthesia monitoring device 102 may be made of or may include one or more materials configured to support using the screenless anesthesia monitoring device 102 during routine patient monitoring or monitoring patient physiological parameters in accordance with the present disclosure. The housing 104 of the screenless anesthesia monitoring device 102 may be configured to protect the screenless anesthesia monitoring device 102 from precipitation (e.g., rain, snow, ice, and sleet), intentional or accidental exposure to hose-directed water or water splashing, airborne dust, sand and debris, corrosion from airborne salt particles or from exposure to chemicals, careless handling by people or animals, impacts from heavy objects or equipment, temporary or prolonged submersion in water, and so on.

The screenless anesthesia monitoring device 102 may be powered by a power source 106, such as, for example, a rechargeable or a non-rechargeable battery. In one example, all active components of the screenless anesthesia monitoring device 102 are powered using one or more batteries, e.g., AA- or AAA-size alkaline or nickel metal hydride rechargeable batteries. Put another way, the screenless anesthesia monitoring device 102 may be configured to operate without an external connection to mains power delivery system, such as, an electrical power grid or another form of high-capacity or long-distance electrical energy transfer and delivery infrastructure. Using battery power may assist in ensuring patient safety and/or obviate a need for galvanic isolation devices and techniques, thus, greatly simplifying the use of the screenless anesthesia monitoring device 102 in remote environments or environments without reliable electrical energy infrastructure in accordance with the present disclosure.

In some examples, the screenless anesthesia monitoring device 102 does not include (i.e., excludes) an integrated display screen, since such integrated display screens may increase the size of diagnostic and monitoring equipment making the equipment significantly less transportable and/or because a display screen of a medical or consumer device or equipment is often exposed and may be one of the most fragile parts of that device or equipment increasing a possibility that the equipment may become inoperable or unusable.

The screenless anesthesia monitoring device 102 is in communication with one or more sensors and may receive one or more signals from each of the sensors. The screenless anesthesia monitoring device 102 monitors and controls operation of the sensors or devices and may be configured to transmit, in raw form or, upon partial or full processing, data received from the sensors to the mobile communication device 130. The screenless anesthesia monitoring device 102 is configured to monitor and collect data indicative of one or more physiological parameters. In one example, the screenless anesthesia monitoring device 102 includes, or communicates with and/or controls operation of, a temperature measurement sensor 108 for determining temperature, an electrocardiogram device 110 for detecting electrocardiogram signals of the patient, an oxygen saturation level measurement device 112 for monitoring the level of oxygen saturation in the blood of the patient, a non-invasive blood pressure measurement device 114 for monitoring of the blood pressure of the patient, and a carbon dioxide measurement device 120 for measuring the level of carbon dioxide (CO2) exhaled by the patient.

Each of the sensors or devices 108, 110, 112, 114, and 120 of the screenless anesthesia monitoring device 102 may be configured to be operatively coupled to the housing 104 of the screenless anesthesia monitoring device 102 via one or more interfaces, connectors, cables, tubes, and/or hoses for attaching to the patient body. Each of the devices 108, 110, 112, 114, and 120 may be communicatively coupled to a processor 126 integral to the screenless anesthesia monitoring device 102 and may be configured to transmit data indicative of detected physiological parameters of a patient's body to the processor 126 on a periodic or a continuous basis. Additionally or alternatively, the processor 126 is configured to monitor and control operation of each of the sensors or devices 108, 110, 112, 114, and 120 and other components of the screenless anesthesia monitoring device 102, such as issuing one or more signals or commands to enable, disable, turn on, turn off, or otherwise alter operation of these devices and components. The processor 126 of the screenless anesthesia monitoring device 102 may be configured to operate in one or more of command and polling modes, and a streaming mode, such as to take advantage of high data speeds available from one or more sensors coupled to or in communication with the screenless anesthesia monitoring device 102.

The screenless anesthesia monitoring device 102 includes a temperature sensor 108. The temperature sensor 108 may be configured to detect one or more parameters to determine temperature of body or skin of a patient in accordance with the present disclosure. The temperature sensor 108 may be contact-based or contactless and may include one or more probes, pads, or other extensions configured to be placed remotely, externally, internally, or via some combination thereof, with respect to the body or skin of the patient. As just one example, the temperature sensor 108 may be one of a thermistor probe, a liquid crystal strip, and an infrared thermometer configured to be placed on or aimed at a forehead of the patient. Other examples of the temperature sensor 108 include, but are not limited to, a negative temperature coefficient (NTC) thermistor, resistance temperature detectors (RTD), thermocouples, and a semiconductor-based temperature sensors. As just one example, a thermistor-type temperature sensor 108 may be configured to detect a continuous, small, incremental change in resistance correlated to variations in temperature, such as detecting a higher resistance at low temperatures and a lower resistance at high temperatures, detecting decrease in resistance as the temperature increases, detecting increase in resistance as the temperature decreases, and so on. As another example, a resistance temperature-type temperature sensor 108 may comprise a film or a wire (platinum, nickel, copper or another material) wrapped around a ceramic or glass core and may be configured to change resistance of the coiled element of the sensor with a detected change in temperature. As still another example, a thermocouple-type temperature sensor 108 may include two wires of different metals electrically bonded at one or more points, such that varying voltage created between these two dissimilar metals corresponds to predefined proportional changes in temperature, i.e., nonlinear measurements interpreted by way of a conversion using a lookup table. As yet another example, a semiconductor-based-type temperature sensor 108 may include one or more integrated circuits (ICs) having one or more diodes with temperature-sensitive voltage versus current characteristics used to monitor changes in temperature.

The screenless anesthesia monitoring device 102 includes an electrocardiogram (ECG or EKG) device 110 for monitoring cardiac rhythm. The electrocardiogram device 110 may include one or more connector cables, probes, and electrical leads configured to be place in one or more locations with respect to one or more organs of the patient's body, such as, but not limited to, heart, lungs, brain, liver, kidney, and so on. In one example, the electrocardiogram device 110 may include a three electrocardiogram connector cables, i.e., a three-lead electrocardiogram device 110. In one example, an analog front-end integrated circuit, attached to the microcontroller via a dedicated serial peripheral interface (SPI) bus, may be used for electrocardiogram signal acquisition. In another example, the electrocardiogram device 110 may be configured to perform 5-lead electrocardiogram detection. In still other examples, the electrocardiogram device 110 may be configured to perform 8- and/or 12-lead electrocardiogram detection.

The electrocardiogram device 110 may be in communication with the processor 126 of the screenless anesthesia monitoring device 102. In an example, electrocardiogram measurement firmware implemented with the electrocardiogram device 110 of the screenless anesthesia monitoring device 102 may be greatly simplified, e.g., as compared to existing electrocardiogram measurement devices. More specifically, the screenless anesthesia monitoring device 102 may be configured to limit operations to controlling and executing analog front-end, including proprietary initialization, calibration, and configuration routines, to collect electrocardiogram data samples, perform noise filtering on the collected data using a FIR software filter, and pass the filtered samples on to the mobile communication device 130 for further processing and analysis. In other words, the screenless anesthesia monitoring device 102 of the present disclosure is configured such that no data processing or analysis takes place within the processor 126 or any other component integral to the screenless anesthesia monitoring device 102.

The screenless anesthesia monitoring device 102 includes a module for measuring blood oxygen saturation level with bidirectional communication. Pulse oximetry to estimate specific oxygen in the bloodstream may be performed using a blood oxygen saturation level measurement device 112 including, as one example, analog front end and supporting circuitry communicatively connected to the processor 126. In an example, the blood oxygen saturation level measurement device 112 includes one or more pulse oximetry finger probes operating via one or more Nelcor-compatible plugs.

Accordingly, the screenless anesthesia monitoring device 102 may be configured to identify levels blood oxygen saturation within and outside one or more predefined thresholds. For example, the screenless anesthesia monitoring device 102 may determine whether the detected level of oxygen saturation is between 95 and 100 percent. In response to the detected level of oxygen saturation is less than or greater than a predefined threshold, the screenless anesthesia monitoring device 102 may be configured to cause the mobile communication device 130 to issue an audible and/or display a visual user alert or notification indicating that the detected level of oxygen saturation is less than or greater than a predefined threshold. Accordingly, without having an integrated screen, the screenless anesthesia monitoring device 102 is configured to issue a notification or command to the mobile communication device 130 to notify medical personnel, including via one or more corresponding website pages 220, that the detected level of oxygen saturation is less than or greater than a predefined threshold.

Pulse oximetry firmware of the screenless anesthesia monitoring device 102 in accordance with the present disclosure utilizes a software-based implementation of a finite impulse response (FIR) filter to remove 50-Hz and 60-Hz mains noise (for example, radiated electrical noise from fluorescent lighting) and may improve accuracy of the captured sensor data in different environments. Furthermore, there is an automatic self-calibration of the LED finger probe for the pulse oximeter; the screenless anesthesia monitoring device 102 may be configured to detect whether the probe is connected to the body of the patient correctly or whether the probe connection is interrupted and/or the probe is removed from the body of the patient. In response to detecting a corresponding connection state, the screenless anesthesia monitoring device 102 may be configured to initiate one or more predefined sensing, computing, or processing operations or routines to use the finger probe for the pulse oximeter to detect being connected to the patient. This helps to remove error induced by different operating conditions, ambient light, patient-to-patient variation, and probe-to-probe variation.

A sensor 114 may be configured to conduct a non-invasive blood pressure measurement 116. In one example, the sensor 116 includes an air pump, one or more valves 118, and sensing circuitry configured to measure blood pressure using, for example, a ratiometric method.

An amount of carbon dioxide exhaled by a patient may be measured with a capnographic system including a medical facemask, a 0.5 liter/minute gas pump 122 and an NDIR-based CO₂ sensor 120 with draw sensing capability. The carbon dioxide measurement device 120 may include a built-in microcontroller that communicates with the processor 126 using a predefined communication protocol, e.g., universal asynchronous receiver-transmitter (UART) communication protocol and so on.

As described above, the screenless anesthesia monitoring device 102 includes the processor 126 configured to perform data aggregation. The processor 126 is further configured to monitor and control operation of each of the devices 108, 110, 112, 114, and 120 and other components of the screenless anesthesia monitoring device 102, such as issuing one or more signals or commands to enable, disable, turn on, turn off, or otherwise alter operation of these devices and components. The processor 126 may be configured to perform noise filtering on at least a portion of the data received from the sensors and/or devices 108, 110, 112, 114, and 120, aggregate filtered and/or unfiltered data into data packets, and cause the data to be transmitted to the mobile communication device 130, e.g., using bi-directional radio protocol for wireless communication 124.

Additionally or alternatively, the screenless anesthesia monitoring device 102 may be configured to transfer sensor data to the mobile communication device 130, such that that mobile communication device 130 performs all complex computation routines, thereby freeing the processor 126 of the screenless anesthesia monitoring device 102 to operate the sensors/devices of the screenless anesthesia monitoring device 102, such as devices 108, 110, 112, 114, and 120 and/or leads, interfaces, and connectors connecting the patient's body to the screenless anesthesia monitoring device 102. In an example, the screenless anesthesia monitoring device 102 includes a communication device 124, such as, but not limited to, a bidirectional wireless/radio module.

The screenless anesthesia monitoring device 102 is entirely self-contained and portable. In some instances, the screenless anesthesia monitoring device 102 does not include, i.e., excludes, an integrated user display interface. Accordingly, in some implementations, the screenless anesthesia monitoring device 102 may include only a power switch and the measurement devices manually connectable to and disconnectable from the screenless anesthesia monitoring device 102 via appropriate interfaces, e.g., plug-and-play interfaces.

The mobile communication device 130 with appropriate software receives the data transmitted from the wireless communication module 124 of the screenless anesthesia monitoring device 102 using a built-in adapter for wireless communication 132 and using application software running on a processor 134 of the mobile communication device 130 performs advanced digital signal processing and waveform analysis of raw patient data, displays (e.g., on a display 138 of the mobile computing device 130 using a graphic adapter card 136 of the mobile communication device 130) patient vital signs data in both graphical and numeric form, handles routine user interface and command and control tasks, and provides alarm functionality to audibly and visually indicate abnormalities in patient vital signs. Patient data is cached in a memory device of the mobile communication device 130. In response to a corresponding signal or request, the mobile communication device 130 and/or the screenless anesthesia monitoring device 102 may be configured to send the data, e.g., using hypertext transfer protocol (HTTP), for storage and analysis on a computer cloud, network drive or other software/hardware configuration enabled for storing or/and processing of data.

FIG. 2 illustrates an example system 200 for monitoring one or more physiological parameters of a patient under a general anesthesia or during routine observation. The system 200 includes the screenless anesthesia monitoring device 102 communicatively coupled, via a network 210, to the mobile communication device 130. While the screenless anesthesia monitoring device 102 and the mobile communication device 130 are illustrated as being communicatively coupled via a network 210, contemplated implementations of the system of the present disclosure is not limited thereto.

The network 210 may be embodied as any type of network capable of communicatively connecting the screenless anesthesia monitoring device 102 and the mobile communication device 130, such as a cloud network, an Ethernet-based network, ad hoc network, etc.

Accordingly, the network 210 may be established through a series of links/interconnects, switches, routers, and other network devices which are capable of connecting the screenless anesthesia monitoring device 102 and the mobile communication device 130 of the network 210. As described in reference to at least FIG. 1, the screenless anesthesia monitoring device 102 and the mobile communication device 130 form communicative coupling to conduct data exchange, data processing, and/or data analysis.

As also described in reference to FIG. 1, the screenless anesthesia monitoring device 102 is configured to receive one or more signals related to detecting, monitoring, and analyzing at least one of a plurality of physiological parameters of a patient under a general anesthesia or during routine observation. To that end, the screenless anesthesia monitoring device 102 may receive signals from one or more sensors, devices, or probes directly, i.e., physically, or indirectly, i.e., wirelessly, coupled to the screenless anesthesia monitoring device 102. As described in reference to at least FIG. 1, the sensors, devices, or probes comprise a temperature sensor for detecting temperature, an electrocardiogram device for detecting electrocardiogram signals from the patient, an oxygen saturation level measurement device for monitoring the level of oxygen saturation in the blood of the patient, a non-invasive blood pressure measurement device for monitoring of the blood pressure of the patient, and a device for measuring the level of carbon dioxide exhaled by the patient.

Once received, the screenless anesthesia monitoring device 102 may relay, transmit, or otherwise transfer the data, either immediately or after a predefined delay, to the mobile communication device 130. In some instances, prior to transmitting the data to the mobile communication device 130, the screenless anesthesia monitoring device 102 may perform one or more operations on the data to compress or encrypt the data to, among other benefits, ensure fast, lossless, and/or secure data transmission. Additionally or alternatively, the screenless anesthesia monitoring device 102 may process all or a portion of the sensor data (e.g., clean, harmonize, organize, prioritize, arrange in a hierarchy, categorize according to one or more attributes, etc.) prior to transmitting the data to the mobile communication device 130. In some other examples, one or more of the sensors, devices, or probes may be configured to transmit data directly to the mobile communication device 130.

Once received, the mobile communication device 130 may perform analytics processes based on, or using, the received physiological parameter data. While not illustrated separately, the mobile communication device 130 may comprise (or be communicatively connected to) a data aggregation service, e.g., that executes on the network 210. The results of the analytics analysis output by the mobile communication device 130 may then be used for various purposes. For instance, the mobile communication device 130 may transmit all or a portion of the received data (or all or a portion of the results of the analytics processes performed on the received data) to be accessible, and, more specifically, to be viewable, via one or more website pages 220.

The mobile communication device 130 may be embodied as any type of electrical, electronic, or electromechanical device capable of performing functions in accordance with the present disclosure, including, but not limited to, a personal communication device, a laptop, a personal digital assistant (PDA), a compute device, a storage device, a server (e.g., stand-alone, rack-mounted, blade, etc.), a sled (e.g., a compute sled, an accelerator sled, a storage sled, etc.), an enhanced network interface controller (NIC), a network appliance (e.g., physical or virtual), a router, a web appliance, a distributed computing system, a processor-based system, and/or a multiprocessor system.

FIG. 3 illustrates an exemplary implementation 300 of the mobile communication device 130. While the illustrated implementation 300 describes only the mobile communication device 130, in other examples, the screenless anesthesia monitoring device 102 may be embodied to include similar components configured to perform similar operations to those described with respect to the mobile communication device 130. The mobile communication device 130 includes a sensor data compute engine 302, an I/O subsystem 308, one or more sensor data storage devices 310, and communication circuitry 312. It will be appreciated that the mobile communication device 130 may include other or additional components, such as those commonly found in a typical computing device (e.g., various input/output devices and/or other components), in other embodiments. Additionally, in some embodiments, one or more of the illustrative components may be incorporated in, or otherwise form a portion of, another component.

The sensor data compute engine 302 may be embodied as any type of device or collection of devices capable of performing the described various compute functions. In some embodiments, the sensor data compute engine 302 may be embodied as a single device, such as an integrated circuit, an embedded system, a field-programmable gate array (FPGA), a system-on-a-chip (SOC), an application-specific integrated circuit (ASIC), reconfigurable hardware or hardware circuitry, or other specialized hardware to facilitate performance of the functions described herein. In some embodiments, the sensor data compute engine 302 may include, or may be embodied as, one or more processors 304 (i.e., one or more central processing units (CPUs), such as the processor 134) and memory 306.

The processor(s) 304 may be embodied as any type of processor capable of performing the described functions. For example, the processor(s) 304 may be embodied as one or more single-core processors, one or more multi-core processors, a digital signal processor, a microcontroller, or other processor or processing/controlling circuit(s). In some embodiments, the processor(s) 304 may be embodied as, include, or otherwise be coupled to an FPGA, an ASIC, reconfigurable hardware or hardware circuitry, or other specialized hardware to facilitate performance of the described functions.

The memory 306 may be embodied as any type of volatile (e.g., dynamic random access memory (DRAM), etc.) or non-volatile memory or data storage capable of performing the described functions. It will be appreciated that the memory 306 may include main memory (i.e., a primary memory) and/or cache memory (i.e., memory that can be accessed more quickly than the main memory). Volatile memory may be a storage medium that requires power to maintain the state of data stored by the medium. Non-limiting examples of volatile memory may include various types of random access memory (RAM), such as DRAM or static random access memory (SRAM).

The sensor data compute engine 302 is communicatively coupled to other components of the mobile communication device 130 via the I/O subsystem 308, which may be embodied as circuitry and/or components to facilitate input/output operations with the processor 304, the memory 206, and other components of the mobile communication device 130. For example, the I/O subsystem 308 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O subsystem 308 may form a portion of a system-on-a-chip (SoC) and be incorporated, along with the sensor data compute engine 302 (e.g., the processor 304, the memory 306, etc.) and/or other components of the mobile communication device 130, on a single integrated circuit chip.

The one or more sensor data storage devices 310 may be embodied as any type of storage device(s) configured for short-term or long-term storage of data, such as, for example, memory devices and circuits, memory cards, hard disk drives, solid-state drives, or other data storage devices. Each sensor data storage device 310 may include a system partition that stores data and firmware code for the data storage device 310. Each sensor data storage device 310 may also include an operating system partition that stores data files and executables for an operating system.

The communication circuitry 312 may be embodied as any communication circuit, device, or collection thereof, capable of enabling communications between the mobile communication device 130 and other computing devices, such as the screenless anesthesia monitoring device 102, the website 220, etc., as well as any network communication enabling devices, such as a gateway, an access point, other network switches/routers, etc., to allow ingress/egress of network traffic. Accordingly, the communication circuitry 312 may be configured to use any one or more communication technologies (e.g., wireless or wired communication technologies) and associated protocols (e.g., Ethernet, Bluetooth®, WiMAX, LTE, 5G, etc.) to effect such communication.

It should be appreciated that, in some embodiments, the communication circuitry 312 may include specialized circuitry, hardware, or combination thereof to perform pipeline logic (e.g., hardware algorithms) for performing the functions described herein, including processing network packets (e.g., parse received network packets, determine destination computing devices for each received network packets, forward the network packets to a particular buffer queue of a respective host buffer of the compute device 102, etc.), performing computational functions, etc.

In some embodiments, performance of one or more of the functions of the described communication circuitry 312 may be performed by specialized circuitry, hardware, or combination thereof of the communication circuitry 312, which may be embodied as a system-on-a-chip (SoC) or otherwise form a portion of a SoC of the compute device 102 (e.g., incorporated on a single integrated circuit chip along with a processor 304, the memory 306, and/or other components of the compute device 102). Alternatively, the specialized circuitry, hardware, or combination thereof may be embodied as one or more discrete processing units of the compute device 102, each of which may be capable of performing one or more of the described functions.

An advantage of the disclosed anesthesia monitoring system over prior art systems is that, while the microcontroller manages the data collection process from the sensors and some rudimentary signal processing, more advanced digital signal processing functions are performed in the mobile device which has much more computational capacity. Separation of data collection from signal processing frees up the limited microcontroller resources to enable it to collect data at higher rates, increasing the quality of the collected data. A further advantage of this separation is that as future improvements and refinements are made to the signal processing algorithms, and as mobile devices have increasing computational capabilities, the algorithms are easily upgradable without requiring reflashing embedded firmware in the screenless anesthesia monitoring device.

Taking in consideration the capabilities of mobile devices the disclosed solution does not exclude embodiments that would use application software that transmit data to remote locations using HTTP or other protocols, where the data can be analyzed or stored for recordkeeping purposes.

While the concepts of the present disclosure are susceptible to various modifications and alternative forms, specific exemplary embodiments are been shown by way of example in the drawings and will be described. It should be understood, however, that there is no intent to limit the concepts of the present disclosure to the particular forms disclosed; on the contrary, the intention is to cover all modifications, equivalents, and alternatives falling within the spirit and scope of the invention as defined by the appended claims.

References in the specification to “one embodiment,” “an embodiment,” “an illustrative embodiment,” etc., indicate that the described embodiment may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C): (A and B); (B and C); (A and C); or (A, B, and C). Similarly, items listed in the form of “at least one of A, B, or C” can mean (A); (B); (C): (A and B); (B and C); (A and C); or (A, B, and C).

The disclosed embodiments may be implemented, in some cases, in hardware, firmware, software, or any combination thereof. The disclosed embodiments may also be implemented as instructions carried by or stored on one or more transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

1. A data collecting device comprising:
a microcontroller communicatively coupled to at least one of a plurality of sensors, the sensors being configured to detect vital signs of a patient including at least one of: cardiac rhythm, temperature, blood oxygen saturation level, blood pressure, and a level of carbon dioxide exhaled by the patient,
the microcontroller being configured to, in response to receiving data from at least one of the sensors, perform noise filtering and aggregate the received sensor data into a plurality of data packets, and transmit the filtered and aggregated data to portable mobile computing device communicatively coupled thereto.

2. The device of claim 1, further comprising a power source comprising one or more alkaline cells.

3. The device of claim 1, wherein the microcontroller is configured to cause the aggregated data to be cached in the mobile computing device, and wherein the microcontroller is configured to transfer the received sensor data to at least one of a computer cloud and a network drive.

4. A data collecting device comprising:
a plurality of sensors configured to detect vital signs of a patient including at least one of: cardiac rhythm, temperature, blood oxygen saturation level, blood pressure, and a level of carbon dioxide exhaled by the patient; and
a microcontroller communicatively coupled to at least one of the sensors and being configured to, in response to receiving data from the at least one of the sensors, transmit the received sensor data to a mobile computing device communicatively coupled thereto.

5. The device of claim 4, wherein, prior to transmitting the received sensor data, the microcontroller is configured to filter the received sensor data.

6. The device of claim 5, wherein the microcontroller is configured to filter the received sensor data to remove 50 Hz and 60 Hz mains noise.

7. The device of claim 4, wherein the microcontroller is configured to cause the aggregated data to be cached in the mobile computing device.

8. The device of claim 4, wherein the microcontroller is configured to transfer the received sensor data to at least one of a computer cloud and a network drive.

9. The device of claim 4, further comprising a power source configured to power the microcontroller, wherein the power source is a battery.

10. A method comprising:
detecting, by a microcontroller of a screenless anesthesia monitoring device, a value of at least one vital parameter of a patient including at least one of: cardiac rhythm, temperature, blood oxygen saturation level, blood pressure, and a level of carbon dioxide exhaled by the patient, wherein the microcontroller is communicatively coupled to at least one of a plurality of sensors, and wherein detecting the vital parameters includes receiving signals from the at least one of the plurality of sensors including data indicating the value of the detected vital parameter;
performing noise filtering and aggregate the received sensor data into a plurality of data packets; and
transmitting the filtered and aggregated data to a portable mobile computing device communicatively coupled to the screenless anesthesia monitoring device, wherein a processor of the screenless anesthesia monitoring device is configured to cause the value of the detected vital parameter to be displayed on a website page.

11. The method of claim 10, wherein filtering the received sensor data includes removing 50 Hz and 60 Hz mains noise.

12. The method of claim 10, wherein the processor of the mobile communication device is configured to cache the aggregated data in a storage device of the mobile communication device.

13. The method of claim 10 further comprising transferring the received sensor data to at least one of a computer cloud and a network drive communicatively coupled to at least one of the screenless anesthesia monitoring device and the mobile communication device.

14. The method of claim 10, wherein the microcontroller of the screenless anesthesia monitoring device is powered using a power source, and wherein the power source is a battery.

15. The method of claim 10, wherein the sensors include a corresponding one of a temperature measurement sensor, an electrocardiogram device, an oxygen saturation level measurement device, a non-invasive blood pressure measurement device, and a carbon dioxide measurement device.
